(19) United States
(12) Patent Application Publication
(10) Pub. No.: US 2026/0072011 A1
(43) Pub. Date: Mar. 12, 2026

(54) APPLICATION FOR PIR-HSA-164586 AND MYH9

(71) Applicant: Qingdao Kangmingbei Jian Biopharmaceutical Co., Ltd., Qingdao City (CN)

(72) Inventors: Wenhua XU, Qingdao City (CN); Xiaomin WANG, Qingdao City (CN); Yan TANG, Qingdao City (CN); Xiao XU, Qingdao City (CN); Xiaoying KONG, Qingdao City (CN); Jiaxu SONG, Qingdao City (CN); Mengyu ZHANG, Qingdao City (CN); Xin MA, Qingdao City (CN); Yiwei XU, Qingdao City (CN); Junlin LV, Qingdao City (CN); Nailong PAN, Qingdao City (CN); Zhen SHANG, Qingdao City (CN); Liang ZHANG, Qingdao City (CN); Dan HAN, Qingdao City (CN)

(73) Assignee: Qingdao Kangmingbei Jian Biopharmaceutical Co., Ltd., Qingdao City (CN)

(21) Appl. No.: 19/216,769

(22) Filed: May 23, 2025

(30) Foreign Application Priority Data
Sep. 12, 2024 (CN) 2024112734476

(51) Int. Cl. G01N 33/50; C07K 14/47; G01N 33/539; G01N 33/68

(52) U.S. Cl. CPC G01N 33/5011; C07K 14/4716; G01N 33/5044; G01N 33/539; G01N 33/6848; G01N 2500/02; G01N 2500/10

(57) ABSTRACT

The present invention belongs to the field of biomedical technology, and specifically relates to a new application for piR-hsa-164586 and MYH9, based on the interaction between piR-hsa-164586 and MYH9: piR-hsa-164586 regulates the expression of MYH9 and promotes the metastasis of NSCLC, piR-hsa-164586 and MYH9 can be used as a therapeutic target for NSCLC, and have become a potential therapeutic drug, wherein the MYH9 is obtained by RNA pulldown assay and verified by protein mass spectrometry, molecular docking and RIP assays, the piR-hsa-164586 can positively regulate the expression of MYH9, and verified by performed qRT-PCR, western blotting and tissue immunofluorescence staining assays on the piR-hsa-164586 NC group, piR-hsa-164586 KD group, and piR-hsa-164586 OE group. The interaction between piR-hsa-164586 and MYH9 can up-regulate the expression of MYH9 in NSCLC cell lines, the verification experiment process was confirmed by Transwell experiment after knockdown of piR-hsa-164586 and MYH9 respectively.

### TECHNICAL FIELD

The present invention belongs to the field of biomedical technology, and specifically relates to a new application for piR-hsa-164586 and MYH9 (myosin heavy chain 9), the progression of NSCLC (non-small cell lung cancer) is promoted by promoting the migration of A549 cells, as a therapeutic target for NSCLC.

### BACKGROUND OF THE INVENTION

The expression of piR-hsa-164586 in serum exosomes of NSCLC patients is up-regulated in serum and cells, which is of great value in the early diagnosis of lung cancer. Additionally, piR-hsa-164586 is localized in the nucleus, which can promote the proliferation, metastasis and angiogenesis of NSCLC cells, but inhibit their apoptosis. Based on this, the mechanism of action of piR-hsa-164586 may provide new ideas for the treatment of NSCLC, the non-coding small RNA located in the nucleus can participate in cancer progression by binding to proteins or RNA to form complexes. In order to further study the role of piR-hsa-164586 in NSCLC, firstly, the protein related to piR-hsa-164586 was obtained by RNA pulldown assay, secondly, protein mass spectrometry analysis, RIP assay and molecular docking were carried out, it was found and proved that a protein named MYH9 (myosin heavy chain 9) interacted with piR-hsa-164586 and was related to the occurrence of NSCLC.

MYH9 is involved in biological processes including cell migration, invasion, adhesion, cytokinesis and polarization, cell shape maintenance and signal transduction, the abnormal expression of MYH9 is closely related to the progression of various cancers, for example, MYH9-dependent polarization promotes rectal cancer metastasis by accelerating focal adhesion assembly; MYH9 can promote the progression of hepatocellular carcinoma by enhancing cancer stemness; MYH9 affects stem cell-like properties in NSCLC by activating signal transduction of AKT-mTOR.

PIWI-interacting RNA (piRNA) is a class of small non-coding RNA (ncRNA) with a length range of 24-31 nucleotides, it typically binds to the piwi protein family to play a regulatory role, piRNA was originally found to play an important role in the reproductive system, and the recent studies have shown that it is closely related to tumorigenesis. Additionally, there is increasing evidence that the abnormal expression of certain piRNAs may be involved in tumorigenesis and is associated with the prognosis of cancer. For example, piRNA-14633 can promote the progression of cervical cancer cells through a MettL14-dependent m6ARNA methylation manner; the abnormal expression of piRNA-54265 in serum can be used as a new biomarker for the early detection and diagnosis of human colorectal cancer.

When exploring the interaction between piRNA and MYH9: firstly, the protein binding to piR-hsa-164586 was obtained by RNA pulldown assay, and the protein profiling was analyzed; secondly, the binding relationship between piR-hsa-164586 and target protein MYH9 was verified by RNA binding protein immunoprecipitation (RIP) assay, the existence of binding site between piR-hsa-164586 and target protein MYH9 was proved by molecular docking assay; finally, the regulating effect of piR-hsa-164586 on downstream target proteins was verified by Quantitative Reverse Transcription Polymerase Chain Reaction (qRT-PCR), western blotting and tissue immunofluorescence staining assays based on mRNA level and protein level, MYH9 and piR-hsa-164586 can play a synergistic effect on the migration of A549 cells by transwell complementation assay.

Hereby, it can be explained that piR-hsa-164586 can positively regulate the expression of MYH9; piR-hsa-164586 can play a synergistic effect with MYH9 to promote NSCLC progression by promoting the migration of A549 cells; piR-hsa-164586 and MYH9 can be used as potential therapeutic targets for NSCLC and have the potential to become new therapeutic drugs.

### SUMMARY OF THE INVENTION

A new application for piR-hsa-164586 and MYH9 in this present invention is based on piR-hsa-164586 promoting NSCLC metastasis by regulating the expression of MYH9 as a therapeutic target for NSCLC.

The MYH9 in this present invention is obtained by RNA pulldown assay and verified by protein mass spectrometry, molecular docking and RIP assays.

The piR-hsa-164586 in this present invention can positively regulate the expression of MYH9, and verified by performed qRT-PCR, western blotting and tissue immunofluorescence staining assays on the piR-hsa-164586 normal control (NC) group, piR-hsa-164586 knockdown (KD) group and piR-hsa-164586 overexpression (OE) group.

The interaction between piR-hsa-164586 and MYH9 in this present invention can up-regulate the expression of MYH9 in NSCLC cell lines, the verification experiments are as follows:
after knockdown of piR-hsa-164586 and MYH9, transwell assay confirmed that the migration number of NSCLC cells is significantly inhibited,, and knockdown of piR-hsa-164586 and MYH9 can further inhibit the migration number of A549 cells;
overexpression of piR-hsa-164586 can significantly increase the migration number of A549 cells, meanwhile, after knockdown of MYH9, the migration number of A549 cells is significantly decreased;
the results show that the binding of piR-hsa-164586 and MYH9 can promote the migration of A549 cells and increase the metastasis ability of NSCLC.

In the present invention, compared with the existing technology, based on the interaction between piR-hsa-164586 and MYH9: piR-hsa-164586 regulates the expression of MYH9 and promotes the metastasis of NSCLC. piR-hsa-164586 and MYH9 can be used as a therapeutic target for NSCLC, and have become a potential therapeutic drug.

### BRIEF DESCRIPTION OF THE DRAWINGS

FIG. 1 is a representative peptide fragment of MYH9 protein for protein profiling analysis according to the present invention.

FIG. 2 is a WB diagram of MYH9 protein pulled down by piR-hsa-164586 after RNA pulldown operation according to the present invention.

FIG. 3 is a schematic diagram of the interaction between piR-hsa-164586 and MYH9 shown by molecular docking according to the present invention.

FIG. 4 is a schematic diagram of qRT-PCR results of MYH9 co-precipitated piR-hsa-164586 according to the present invention, (n=3), *P [symbol] 0.05.

FIG. 5 is a schematic diagram of the verification of the regulating effect on MYH9 by piR-hsa-164586 at the mRNA level by the qRT-PCR technique according to the present invention, (n=3).

FIG. 6A and FIG. 6B shows the results of western blotting wherein FIG. 6A were target protein bands of piR-hsa-164586 based on the regulating effect on MYH9 at the protein level and FIG. 6B were western blotting quantitative results (n=3).

FIG. 7A and FIG. 7B shows an immunofluorescence schematic diagram, wherein FIG. 7A shows an immunofluorescence schematic diagram. (×400 times, scalebar=50 82 μm) of the regulating effect on MYH9 by piR-hsa-164586 on MYH9 according to the present invention and FIG. 7B was an immunofluorescence quantitative analysis diagram (n=3). * P<0.05, * * *P<0.001.

FIG. 8A and FIG. 8B was Transwell experimental, wherein FIG. 8A shows a Transwell experimental results diagram of the effect of the combination of piR-hsa-164586 and MYH9 on cell migration according to the present invention; ns: no significance and FIG. 8B was statistical data diagram, (n=3), *P<0.05, **P<0.01.

### DETAILED DESCRIPTION OF THE INVENTION

The present invention will be further elaborated hereafter in conjunction with accompanying drawings and specific embodiments.

### Embodiment 1

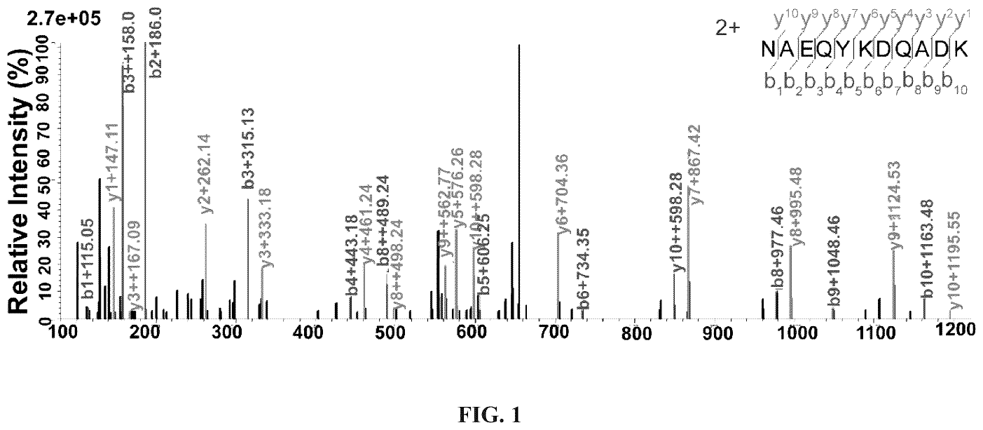

The protein profiling obtained from the protein profiling analysis according to this embodiment as shown in FIG. 1 shows that MYH9 includes five peptide fragments: QLLQANPILEAFGNAK, VASHLLGINVTDFTR, DLGEELEALKTELEDTLDSTAAQQELR, NTDQASMPDNTAAQK and NAEQYKDQADK, which have the greatest binding potential with piR-hsa-164586 and are associated with lung carcinogenesis.

### Embodiment 2

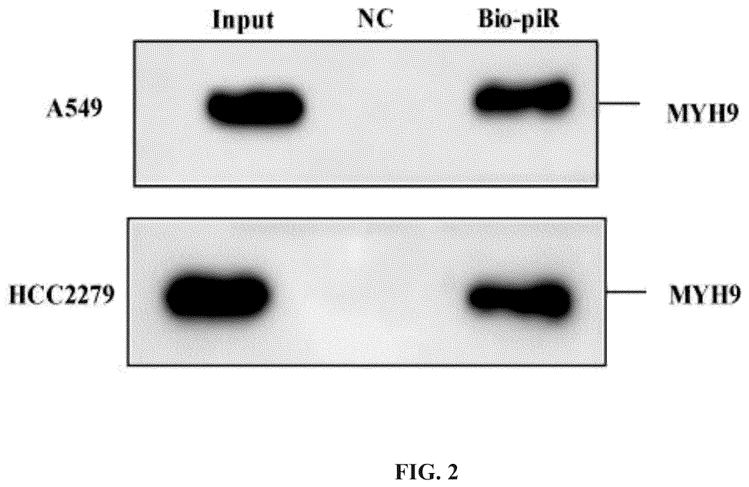

The dissociation process of piR-hsa-164586 binding protein by RNA pulldown technology according to this embodiment is as follows:
(1) two groups of cells (two 10 cm dishes/group) were prepared and rinsed with polybutylene succinate (PBS) 2-3 times, each dish was added with 2 mL of cell lysate, and the cells were scraped off with a cell brush and transferred to 2 mL Ep tube (stainless steel electrolytic polishing tube), and the auto-focusing ultrasound sample processing system was used to lyse and break cells;
(2) centrifuged for 40 min at a speed of 12000 rpm, and an appropriate amount of supernatant was taken as Input;
(3) the probe was prepared to make a final concentration of 10μM;
(4) magnetic separation was performed;
(5) 1 mL Buffer1 was added, and step (4) was repeated;
(6) 500μL of biotinylated nucleic acid diluted with Buffer1 was added, after fully oscillating the re-suspended magnetic beads, and the mixture was fully mixed with a rotating vortex instrument;
(7) the mixture was separated, the supernatant was transferred, washed 3 times, and the WB diagram of MYH9 protein pulled down by piR-hsa-164586 after RNA pulldown operation is shown in FIG. 2.

### Embodiment 3

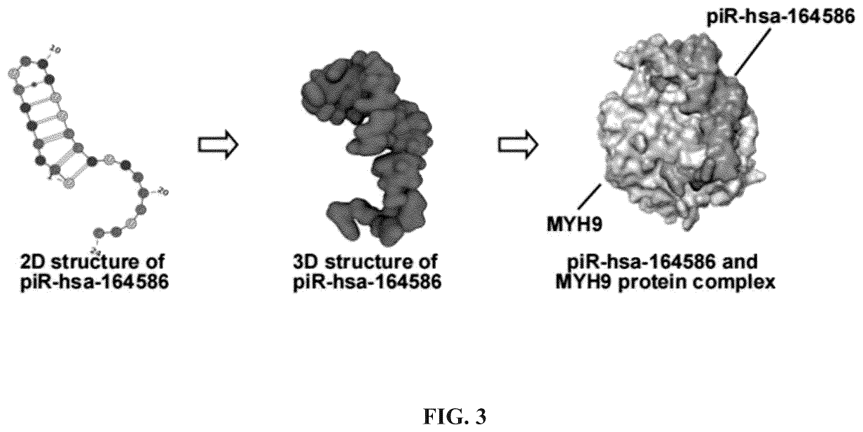

The process of the interaction between piR-hsa-164586 and MYH9 was proved by molecular docking according to this embodiment is as follows:
(1) the secondary structure of piRNA-hsa-164586 was predicted by Vflod2 D;
(2) the tertiary structure of piRNA-hsa-164586 was predicted by Vflod3 D;
(3) the molecular docking was performed by using the GRAMM website, and the results are shown in FIG. 3.

### Embodiment 4

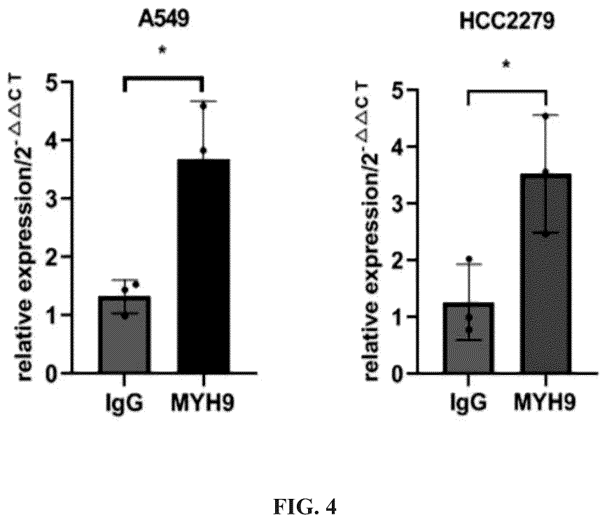

The process of RNA-binding protein aggregation and testing by RIP assay according to this embodiment is as follows:
(1) Buffer A operating fluid was added to the cells and lysed on the ice, vortexed and mixed once every 10 minutes;
(2) centrifugation (4° C., 14000 g, 10 min), the supernatant was retained;
(3) 100 μL was taken from the supernatant as Input;
(4) magnetic beads were added to the remaining supernatant and rotated uniformly;
(5) the supernatant was separated from the magnetic beads and the supernatant was retained;
(6) the magnetic beads, the antibody of the target protein and the internal reference (IgG) and BufferA were mixed respectively, and the mixture was rotated and mixed for 2 h at a temperature of 4° C.;
(7) magnetic separation was performed, and the supernatant was removed;
(8) washing and magnetic separation were performed, and the supernatant was removed;
(9) 1×BufferA and cell lysate were added to the mixed magnetic beads (including the antibody of the target protein and IgG), and placed in a refrigerator at 4° C. for rotation overnight at a low speed (10-15 rpm);
(10) magnetic separation was performed, magnetic beads were retained, and 1 mL 1×BufferB was added to the mixed magnetic beads to mix, separate, and repeat the operation 5 times;
(11) BufferE was added to different groups of mixed magnetic beads (including pre-retained Input tubes), mixed and vortexed, magnetic separation was performed, and the supernatant was retained;
(12) the DNA was removed from the DR Columns filter column (14000 g, centrifuged for 2 min), the filtrate was collected, and 70% ethanol (equal volume with the filtrate) was added to the filtrate, pipetted and mixed;
(13) RNA was purified and filtered in the RC Columns filter column (12000 g, centrifuged for 30-60 s), and 500 μL of Buffer F (containing ethanol) and Buffer G (containing ethanol) were added to the RC Columns filter column and centrifuged for 3 times (12000 g, 2 min/time), each time the filtrate was removed; an appropriate amount of non-RNA enzyme water was added into the center of the transferred new non-RNA enzyme centrifuge tube membrane; dissolved at room temperature for 2 min; RNA was collected (12000 g, centrifuged for 1 min);
(14) the product was verified by qrt-pCR, and the results are shown in FIG. 4.

### Embodiment 5

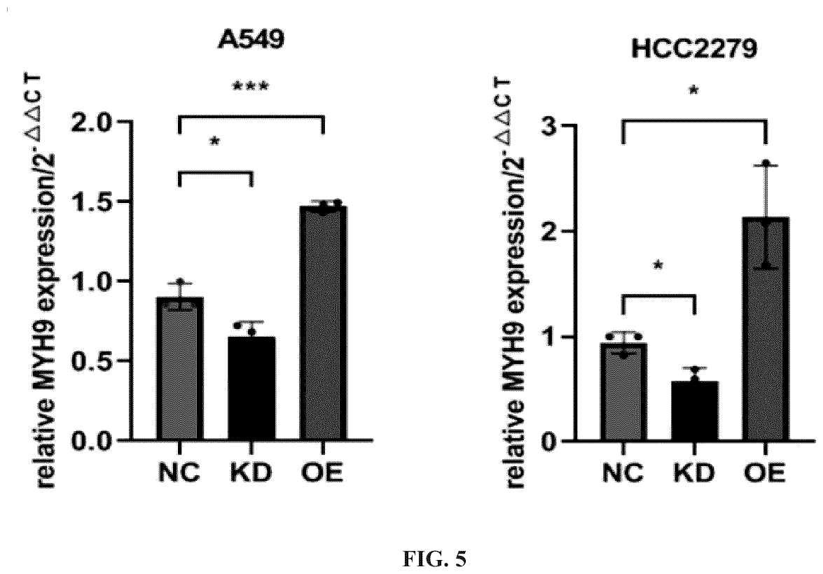

The process of the verification of the regulating effect of piR-hsa-164586 on downstream target proteins by qRT-PCR based on mRNA level according to this embodiment is as follows:
(1) MYH9-related primers was designed;
(2) through the verification of qRT-PCR, the results are shown in FIG. 5.

### Embodiment 6

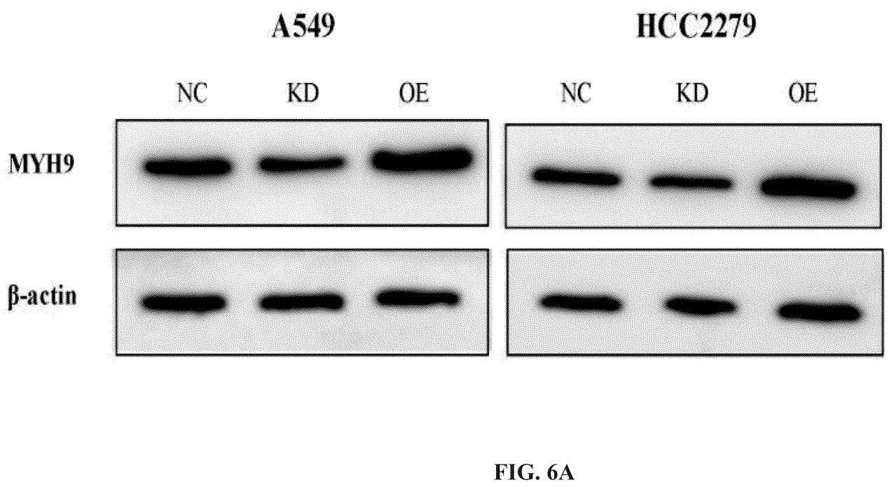

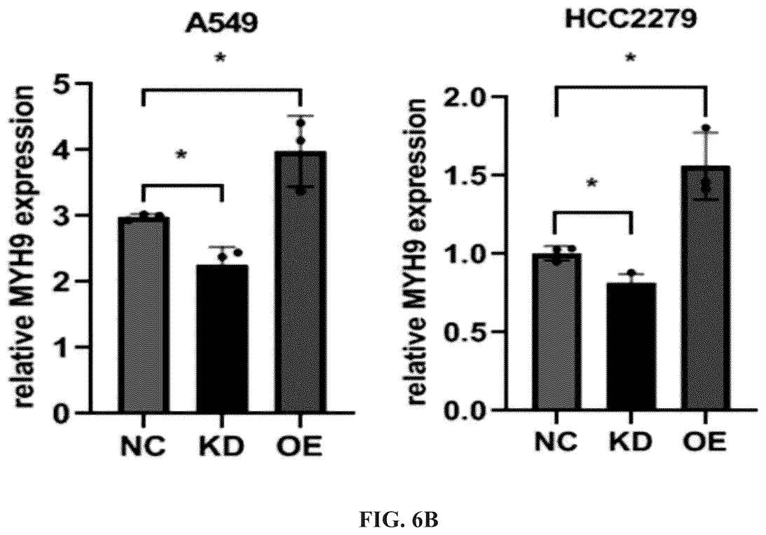

The process of the verification of the regulating effect of piR-hsa-164586 on downstream target proteins by Western blotting based on protein level according to this embodiment is as follows:
(1) the appropriate amount of RIPA (containing 1% PMSF) lysate was added to the extracted and purified exosomes, and lysed on ice for about 30 min to obtain the sample;
(2) the samples were centrifuged at 4° C. in a centrifuge (12000 rpm, 15 min);
(3) the supernatant was collected, and a 5:1 loading buffer was added. The mixture was placed in a metal bath at 95° C. for 5 min, it was used for electrophoresis experiments after cooling;
(4) firstly, the 1.0 mm of WB special glass plate was cleaned with detergent, secondly rinsed with tap water, and finally rinsed with double distilled water and dry;
(5) filled with double distilled water and observed for 5 min, if the liquid level does not decrease significantly, subsequent experiments will be carried out;
(6) 5-6 ml underlayer glue was added;
(7) anhydrous ethanol was added until the liquid surface was flattened, placed at room temperature for about 15 min, and anhydrous ethanol was poured after the underlayer glue was solidified;
(8) the concentrated gel was prepared and placed in a suitable comb, the comb was slowly pulled out after the underlayer glue was solidified, and the 1 protein electrophoresis buffer was poured into the electrophoresis tank for subsequent electrophoresis;
(9) the upper protein sample was run for 30 min under the condition of 80V, and then turned into 120V for 90 min when the sample ran to the upper and lower boundary line, until the end of electrophoresis;
(10) the Polyvinylidene fluoride (PVDF) membrane was cut to match the size of the gel;
(11) the PVDF membrane was activated with methanol and tightly adhered to the gel surface;
(12) the transfer membrane device was placed in the transfer membrane tank, and the ice box was added to the transfer membrane tank and then placed in the foam box with ice;
(13) the membrane was transferred at a constant current of 300 mA, and the transfer time (min) was approximately equal to the molecular weight of the protein;
(14) the transferred PVDF membrane was sealed with a rapid sealing solution at 25 rpm for 20 min at room temperature;
(15) the PVDF membrane was placed in a diluent containing primary antibody and incubated at room temperature for 2 h at a speed of 25 rpm on a shaker or overnight in a refrigerator at 4° C. (12 h);
(16) the membrane was washed with 1×Tris Buffered Saline with Tween-20 (TBST), 50 rpm, 10 min/time, a total of 3 times;
(17) 5% skimmed milk powder was prepared with 1×TBST to dilute secondary antibody, and incubated at room temperature for 1.5 h.
(18) step (3) was repeated;
(19) the membrane was soaked in chemiluminescence solution after absorbing water, and exposed and developed in a chemiluminescence instrument, the results are shown in FIG. 6.

### Embodiment 7

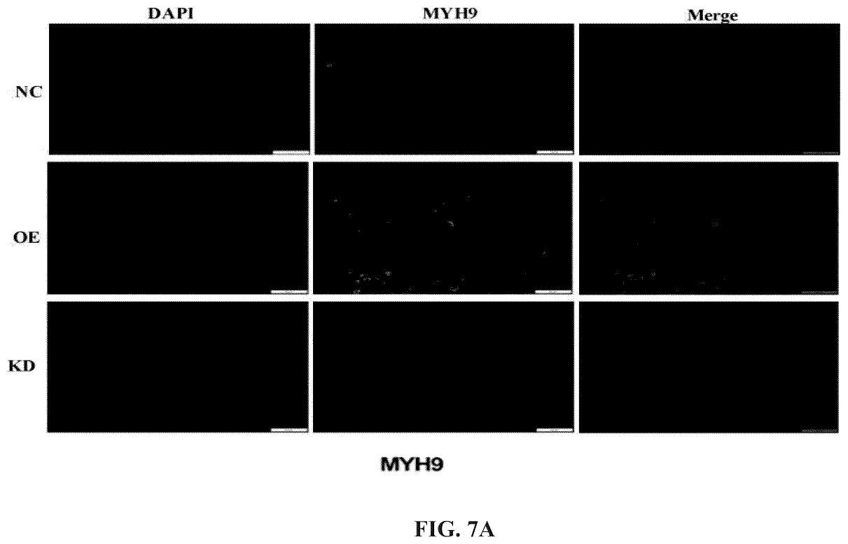

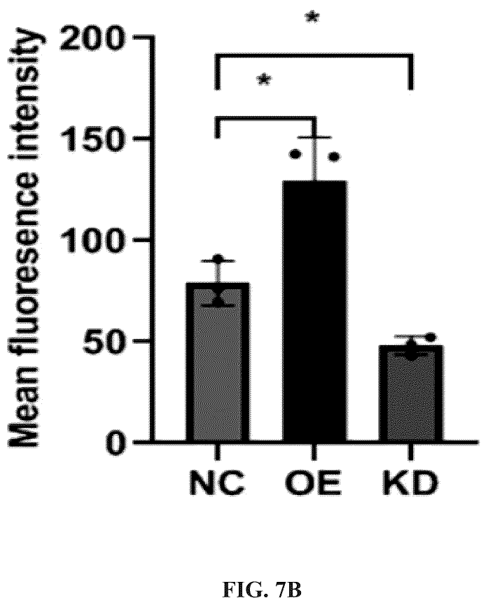

The process of the verification of the regulating effect of piR-hsa-164586 on downstream target proteins based on protein level by immunofluorescence technique according to this embodiment is as follows:
(1) tissue frozen sections were prepared;
(2) tissue frozen sections were fixed with 4% paraformaldehyde for 20 min;
(3) tissue frozen sections were rinsed with PBS buffer 3 times;
(4) tissue frozen sections were blocked with antibody-blocking solution at room temperature for 1 h;
(5) primary antibody was incubated, and incubated overnight at 4° C. protected from light;
(6) the primary antibody was rinsed with PBS 2-3 times, 3-5 min each time;
(7) the secondary antibody was incubated at room temperature for 2 h;
(8) the secondary antibody was rinsed and 4′,6-diamidino-2-phenylindole (DAPI) was used to stain the nucleus;
(9) images were observed and obtained under an inverted fluorescence microscope;
(10) statistical analysis was performed by GraphPad Prism8.0 software (GraphPad Software, USA). As shown in FIG. 7, the fluorescence signal (red) of MYH9 was significantly enhanced after piR-hsa-164586 (P<0.05), and the fluorescence signal of MYH9 was significantly weakened after knockdown of piR-hsa-164586 (P<0.05), indicating that piR-hsa-164586 has a positive regulating effect on MYH9.

### Embodiment 8

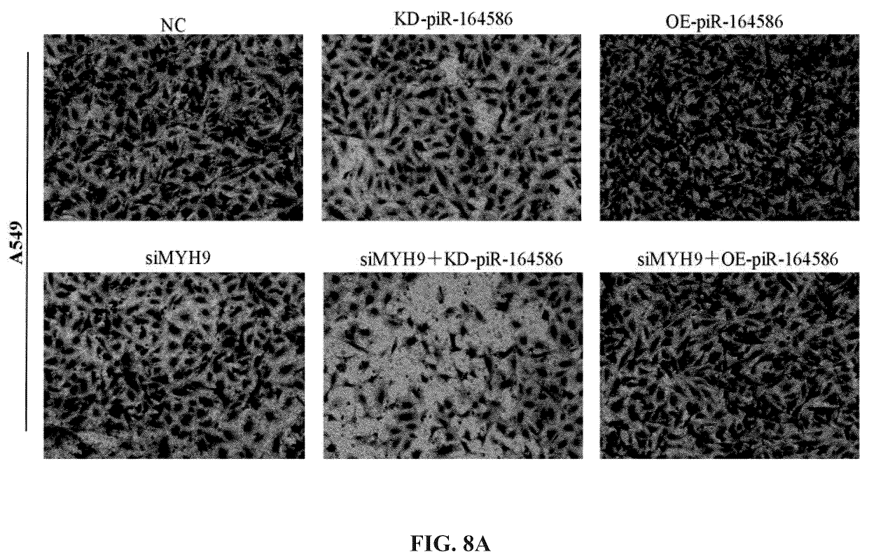

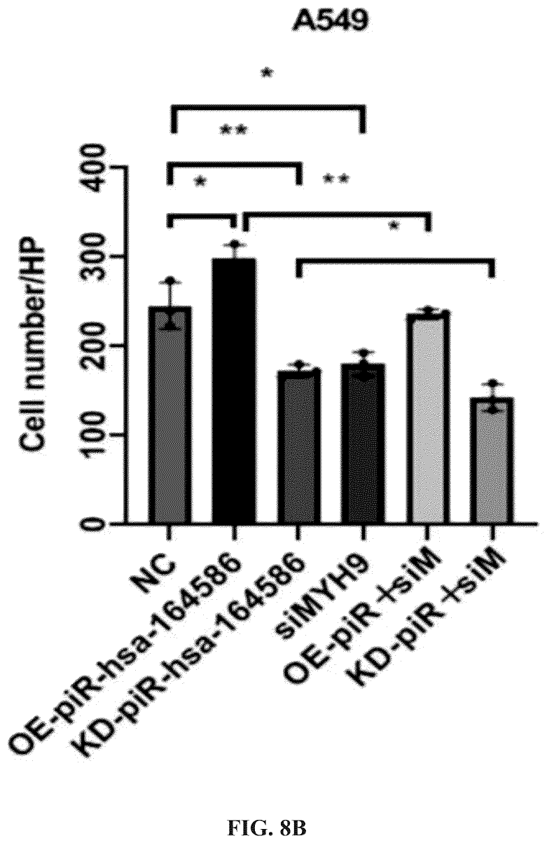

The process of comparing the strength of the migration ability of different groups of cells by Transwell experiment according to this embodiment is as follows:
(1) the NSCLC cell group was divided into: the piR-hsa-164586 NC group, piR-hsa-164586 OE group, piR-hsa-164586 KD group, small interfering MYH9 (siMYH9) group, piR-hsa-164586 OE+siMYH9 group, piR-hsa-164586 KD+siMYH9 group;
(2) 1×105 cells per well were seeded in 12-well plates, and the nucleic acid analogs and inhibitors of piR-hsa-164586 were transfected into the cells after overnight culture;
(3) after transfection for 24 h, the cells were digested and centrifuged, the cell suspension (serum-free medium resuspension) was added to the upper chamber, and the DMEM medium containing 15% FBS was added to the lower chamber, the cells were cultured in a 37° C. of CO2 incubator for 24 h;
(4) the old culture medium in the upper chamber was pipetted out, rinsed, added formaldehyde (4%) and fixed for 20 min;
(5) washed for 3 times with PBS buffer;
(6) the obtained images were observed under a microscope after staining with 0.1% crystal violet solution for 10 min and cleaning with PBS buffer, as shown in FIG. 8.

When MYH9 was knocked down, the migration number of A549 cells decreased (P<0.05), when MYH9 was knocked down and piR-hsa-164586 was knocked down at the same time, the inhibitory effect was aggravated;

When piR-hsa-164586 was overexpressed, the migration number of cells was significantly increased, and MYH9 was knocked down at the same time, the migration effect was significantly inhibited (P<0.05);

it is indicated that the interaction between piR-hsa-164586 and MYH9 has a regulating effect on the migration ability of A549 cells.

Wherein, the statistical methods in embodiments 3-8 were analyzed by GraphPad Prism8.0 software (GraphPad Software, USA) and SPSS22.0 software (IBM, Germany). The normality test of data distribution was evaluated by Kolmoorov-smirnov, if the data conformed to the normal distribution, the t test was selected between the two groups for analysis;

if does not conform to the normal distribution, the Wilcoxon test was selected for analysis;

ANOVA analysis was selected for data analysis of more than two groups;

Image J (V1.8.0.112) was used for image processing, and the ROC curve was constructed to evaluate the diagnostic performance, P<0.05 was considered statistically significant.

All tests were two-tailed tests.

What is claimed is:

1. A new application for piR-hsa-164586 and MYH9, wherein the piR-hsa-164586 and MYH9 as a therapeutic target for NSCLC.

2. The new application for piR-hsa-164586 and MYH9 according to claim 1, wherein a principle is that piR-hsa-164586 promotes NSCLC metastasis by regulating the expression of MYH9.

3. The new application for piR-hsa-164586 and MYH9 according to claim 1, wherein the MYH9 is obtained by RNA pulldown assay and verified by protein mass spectrometry, molecular docking and RIP assays.

4. The new application for piR-hsa-164586 and MYH9 according to claim 1, wherein the piR-hsa-164586 positively regulates the expression of MYH9, and verified by performed qRT-PCR, western blotting and tissue immunofluorescence staining assays on the piR-hsa-164586 NC group, piR-hsa-164586 KD group and piR-hsa-164586 OE group.

5. The new application for piR-hsa-164586 and MYH9 according to claim 1, wherein the interaction between piR-hsa-164586 and MYH9 up-regulates the expression of MYH9 in NSCLC cell lines, the verification experiments as follows:
after knockdown of piR-hsa-164586 and MYH9, Transwell assay confirmed that the migration number of NSCLC cells is inhibited, and the knockdown of piR-hsa-164586 and MYH9 can further inhibit the migration number of A549 cells;
overexpression of piR-hsa-164586 can increase the migration number of A549 cells, meanwhile, after knockdown of MYH9, the migration number of A549 cells is decreased;
the results show that the binding of piR-hsa-164586 and MYH9 can promote the migration of A549 cells and increase the metastasis ability of NSCLC.

6. The new application for piR-hsa-164586 and MYH9 according to claim 2, wherein the MYH9 is obtained by RNA pulldown assay and verified by protein mass spectrometry, molecular docking and RIP assays.

7. The new application for piR-hsa-164586 and MYH9 according to claim 2, wherein the piR-hsa-164586 positively regulates the expression of MYH9, and verified by performed qRT-PCR, western blotting and tissue immunofluorescence staining assays on the piR-hsa-164586 NC group, piR-hsa-164586 KD group and piR-hsa-164586 OE group.

8. The new application for piR-hsa-164586 and MYH9 according to claim 2, wherein the interaction between piR-hsa-164586 and MYH9 up-regulates the expression of MYH9 in NSCLC cell lines, the verification experiments as follows:
after knockdown of piR-hsa-164586 and MYH9, Transwell assay confirmed that the migration number of NSCLC cells is inhibited, and the knockdown of piR-hsa-164586 and MYH9 can further inhibit the migration number of A549 cells;
overexpression of piR-hsa-164586 can increase the migration number of A549 cells, meanwhile, after knockdown of MYH9, the migration number of A549 cells is decreased;
the results show that the binding of piR-hsa-164586 and MYH9 can promote the migration of A549 cells and increase the metastasis ability of NSCLC.
